(19) United States
(12) Patent Application Publication
(10) Pub. No.: US 2013/0091604 A1
(43) Pub. Date: Apr. 11, 2013

(54) ABOVEGROUND ORGAN SPECIFIC PROMOTERS FOR TRANSFORMING PLANTS AND USES THEREOF

(71) Applicant: MYONGJI UNIVERSITY INDUSTRY AND ACADE, Gyeonggi-do (KR)

(72) Inventors: Ju Kon Kim, Seongnam- si (KR); Su Hyun Park, Yongin-si (KR)

(73) Assignee: MYONGJI UNIVERSITY INDUSTRY AND ACADEMIA COOPERATI, Gyeonggi-do (KR)

(21) Appl. No.: 13/647,112

(22) Filed: Oct. 8, 2012

Related U.S. Application Data
(63) Continuation-in-part of application No. PCT/KR2011/002274, filed on Apr. 1, 2011.

(30) Foreign Application Priority Data
Apr. 9, 2010 (KR) 10-2010-0032942

(51) Int. Cl. C12N 15/82

(52) U.S. Cl. CPC C12N 15/8216
USPC 800/288; 536/24.1; 435/320.1; 530/351; 800/298; 530/399; 530/385; 530/356; 530/303; 530/363; 530/380; 530/353

(57) ABSTRACT

A promoter for transformation of a plant, in particular an aboveground organ specific promoter, a recombinant plant expression vector including the promoter, a method of producing target protein using the recombinant plant expression vector, target protein produced by the method, a method of producing a transformed plant using the recombinant plant expression vector, a transformed plant produced by the same, and a seed of the plant.

### RELATED APPLICATION DATA

This application is a continuation-in-part of International Patent Application No. PCT/KR2011/002274 filed on Apr. 1, 2011 and published in the English language, and which claims priority of Korean Patent Application No. 10-2010-0032942 filed on Apr. 9, 2010, all of which are hereby incorporated herein by reference in their entirety.

### TECHNICAL FIELD

The present disclosure relates to aboveground organ specific promoters and a method of producing the same. More particularly, the present disclosure relates to aboveground organ specific promoters for transforming monocotyledonous plants and a method of producing the same.

### BACKGROUND

A promoter is a region positioned on a chromosome at a position upstream of a structural gene and regulates transcription of the downstream structural gene into mRNA. A variety of general transcription factors bind to the promoter, thus becoming activated. Promoters often have a common base sequence structure such as a TATA box, CAT box and the like.

Since the cellular concentration of proteins involved in biological metabolism needs to be kept uniform, genes to which the promoters are linked are only transcribed when transcription factors bind to the promoters. On the contrary, in proteins which are only needed under specific circumstances, inducible promoters are connected to corresponding structural genes to induce expression of the structural genes. That is, specific transcription factors activated by external stimuli related to growth of an organism or environmental factors bind to the inducible promoters to make the promoters active.

In producing agricultural plants having novel characteristics, expression of a transgene to be introduced into a plant body is considerably affected by transcriptional, post-transcriptional, translational, and post-translational elements. Among these elements, a promoter pertaining to the transcriptional elements may directly affect transcription of a transgene, thereby ultimately changing an expression level. Further, the promoter is the most important element, which changes stages of expressing a transgene, or tissue- or cell- specificities. To date, although a great number of promoters have been isolated from various plants for expression of a transgene, only a few promoters among them are practically used for plant transformation.

Cauliflower mosaic virus (CaMV) 35S promoter and derivatives thereof are the most widely used in the art, and act as strong promoters in all plant organs. Cauliflower mosaic virus (CaMV) 35S promoter and derivatives thereof exhibit particularly strong promotion in vascular tissues and most root and leaf cells. However, the CaMV 35S promoter exhibits lower activity in monocotyledonous plants, such as rice and the like, than dicotyledonous plants, and does not exhibit any activity in certain cells, such as pollen.

In addition to CaMV 35S, various other promoters originating from dicotyledonous plants have also been used for transformation of monocotyledonous plants, but exhibit lower activity than promoters originating from monocotyledonous plants. Further, ribulose bisphosphate carboxylase/oxygenase small submit (RbcS) promoter found in rice, Actin1 (Act1) promoter found in rice, and Ubi1 promoter found in maize have been investigated as promoters for transformation of monocotyledonous plants. Particularly, Act1 and Ubi1 promoters exhibit relatively high activity in monocotyledonous plants as compared with the CaMV 35S promoter, and thus have been generally used for transformation of monocotyledonous plants.

However, the Ubi 1 promoter exhibits activity in various types of cells but does not function in all plant tissues. Furthermore, although the Ubi1 promoter exhibits strong activity in young roots, activity is remarkably reduced as the plant matures. The Act1 promoter exhibits activity mainly in elongating tissues and reproductive tissues, and thus is not effective for ubiquitous gene expression in monocotyledonous plants. Thus, there is a need for a promoter exhibiting strong, stable, and ubiquitous activity in monocotyledonous plants.

Therefore, the inventors of the present disclosure have made a strong effort to develop effective promoters for transformation of monocotyledonous plants. As a result, the inventors have found a rice-derived promoter that is well-suited to expression of genes in aboveground organs of monocotyledonous plants, and came to complete the present disclosure.

### SUMMARY

The present disclosure is directed to providing an effective promoter for transformation of plants.

The present disclosure is also directed to providing a recombinant plant expression vector including the above promoter.

The present disclosure is also directed to providing a method of producing a target protein using the promoter and the recombinant plant expression vector, and protein produced by the same.

The present disclosure is also directed to providing a method of producing a transformed plant using the recombinant plant expression vector, and a transformed plant produced by the same.

The present disclosure is also directed to providing a seed of the transformed plant.

In accordance with one aspect, the present disclosure provides a promoter that includes at least one sequence selected from the group consisting of Sequence Identification Number (SEQ. ID. No.) 1 and SEQ. ID. No. 2.

The present disclosure relates to a rice-derived promoter. More particularly, the promoter may be an aboveground organ specific promoter for transformation of monocotyledonous plants. Further, the promoter may be suitable for expression of aboveground plant organs, for example, leaves.

A promoter having SEQ. ID. No. 1 is referred to as “ribulose bisphosphate carboxylase/oxygenase small subunit 3 (RbcS3),” and a promoter having SEQ. ID. No. 2 is referred to as “Phosphoribulokinase (PRK).”

Two newly isolated types of promoters are expressed more strongly in leaves than leaf-specific promoters, e.g. RbcS1 (GenBank Accession No. D00643). Furthermore, the RbcS3 promoter and the PRK promoter induce much stronger expression than existing maize ubiquitin promoter, and have similar expression efficiency to OsCc1 promoter (GeneBank Accession No. AF399666) in leaves.

In one embodiment, a variant of the above sequence may be included in the scope of the invention. The variant has a different base sequence but has similar functional and immunological characteristics to a base sequence of SEQ. ID. No. 1 or SEQ. ID. No. 2. Specifically, the promoter may include a base sequence that has not less than 70%, preferably not less than 80%, more preferably not less than 90%, and still more preferably not less than 95% homology with the base sequences of SEQ. ID. No. 1 or SEQ. ID. No. 2.

“Percent homology (%)” with respect to a polynucleotide sequence is ascertained by comparing two optimally aligned sequences with a comparison site, and part of the polynucleotide sequence in the comparison site may have an addition or deletion (that is, a gap) as compared with a reference sequence (which does include the is additions or deletions) for optimal alignment of the two sequences. Percent homology (%) is calculated by determining the number of positions where the same nucleotide exists in both sequences to calculate the number of corresponding positions, dividing the number of corresponding positions by the total number of positions of the nucleobase in the comparison site, and finally multiplying the result by 100. The optimal alignment of the sequences for comparison may be realized using known computer software (for example, GAP, BESTFIT, FASTA, and TFAST in the Wisconsin Genetics Software Package, Genetics Computer Group (GCG), 575 Science Dr., Madison, Wis., or BlastN and BlastX available from the National Center for Biotechnology Information) or by direct inspection.

Substantial identity of polynucleotide sequence means that polynucleotides include a sequence having at least 70%, preferably at least 80%, more preferably at least 90%, and still more preferably at least 95% identity. In other words, when two molecules are hybridized with each other under strict conditions, the molecules have substantially the same nucleotide sequence. The strict condition is sequence dependent, and may be different in other cases. Generally, the strict condition is selected to be about 10° C. lower than a melting point Tm with respect to a particular sequence at a preset ionic strength and a predetermined pH. Tm is a temperature at which 50% of a target sequence is hybridized to a completely matching probe at a preset ionic strength and a predetermined pH. Tm of a hybrid, which is a function both of probe length and base composition, may be calculated using information in documents (Sambrook, T. et al., (1989) Molecular Cloning—A Laboratory Manual (2nd edition), Volume 1-3, Cold Spring Harbor Laboratory, Cold Spring). Typically, strict conditions with respect to Southern blotting include washing in 0.2×SSC at 65° C. For a preferred oligonucleotide probe, washing conditions include washing in 6×SSC at about 42° C.

In one embodiment, the promoter may include a complementary sequence to the sequence of SEQ. ID. No. 1 or SEQ. ID. No. 2.

The term “complementary” is widely used in the art and means that a nucleotide or nucleic acid, for example, DNA molecule, has two hybridized or base-paired strands.

In one embodiment, the monocotyledonous plants may include, but are not limited to, rice, barley, wheat, maize, millet, or sorghum.

In accordance with another aspect, the present disclosure provides a recombinant plant expression vector including the promoter according to the embodiments of the present disclosure. An example of the recombinant plant expression vector may include, but is not limited to the vector shown in FIG. 2.

Specifically, the vector includes the promoter of the present disclosure operatively linked to transformed green fluorescence protein (GFP) genes, a protease inhibitor II gene terminator (TPINII), an OsCc1 promoter (Pcytc), an herbicide resistant gene Bar (phosphinothricin acetyltransferase gene), and a nopaline synthase (NOS) terminator (TNOS). Further, the vector is combined with a MAR sequence at a right-border sequence end to minimize variation in expression due to an introduced site into a chromosome, so that activity of only the promoter of the present disclosure is measured.

The term “recombinant” denotes a cell which replicates a heterogeneous nucleic acid or expresses the nucleic acid, a peptide, a heterogeneous peptide, or protein encoded by a heterogeneous nucleic acid. A recombinant cell may express a gene or a gene fragment, which is not naturally found in the cell, in the form of a sense or antisense nucleic sequence. In addition, a recombinant cell may express genes naturally found in the cell but re-introduced into the cells through genetic engineering.

The term “vector” is used herein to refer to a DNA fragment or DNA segments and nucleic acids which are delivered to cells. The vector may be used to replicate DNA and be independently reproduced in a host cell. The terms “delivery system” and “vector” are often used interchangeably. The term “expression vector” means a recombinant DNA molecule including a desired coding sequence or other appropriate nucleic acid sequences which are essential for expression of the operatively-linked coding sequence in a particular host organism. Promoters, enhancers, termination signals, and polyadenylation signals which may be used for a eukaryotic cell are publicly known.

A preferred example of a plant expression vector is a T-plasmid vector which may transfer a portion called a T region into a plant cell when the vector is in a proper host, such as Agrobacterium tumefaciens. Another type of T-plasmid vector, which is commonly used to transfer hybrid DNA sequences (see EP 0116718 B1), is a protoplast capable of producing a genetically altered plant into a genome of which a hybrid DNA is sequence is inserted. A preferred type of the Ti-plasmid vector is a binary vector disclosed in EP 0120516 B1 and U.S. Pat. No. 4,940,838. Another proper vector used to introduce DNA into a plant host may be selected from among virus vectors derived from double stranded plant viruses including CaMV, single stranded viruses, and geminiviruses, for example, a non-complete plant virus vector. Use of these vectors may be advantageous when it is difficult to properly transform a plant host.

An expression vector may preferably include at least one selective marker. The marker is a nucleic acid sequence having properties selected by a typical chemical method and includes all genes that can help to differentiate transformed cells from non-transformed cells. For example, the marker may be herbicide resistance genes, such as glyphosate and phosphinothricin, and antibiotic resistance genes, such as kanamycin, G418, bleomycin, hygromycin, and chloramphenicol, without being limited thereto.

The term “promoter” denotes a DNA region upstream of a structural gene and refers to a DNA sequence to which RNA polymerase binds to initiate transcription. The term “plant promoter” means a promoter which may initiate transcription in a plant cell. The term “constitutive promoter” means a promoter which is active under most environmental conditions and development or cell differentiation states. Since identification of transformants can be performed in various tissues at various stages, the constitutive promoter may be proper in the present disclosure. The constitutive promoter does not limit selection possibility.

As for the terminator, any conventional terminator may be used for the present disclosure, for example, nopaline synthase (NOS), a rice a-amylase RAmyl A terminator, a phaseoline terminator, an octopine gene terminator of Agrobacterium tumefaciens, and the like, without being limited thereto. Terminators are generally known to increase certainty and efficiency of transcription in plant cells. Thus, use of terminators is substantially appropriate in the present disclosure.

In a recombinant plant expression vector in accordance with one embodiment of the present disclosure, the plant expression vector may be produced by operatively linking a target gene, which encodes for a target protein, to a region downstream of the promoter of the present disclosure. Here, “operatively-linked” refers to an element of an expression cassette which functions as a unit to express heterogeneous protein. For example, a promoter operatively linked to a heterogeneous DNA sequence which codes for a protein promotes production of functional mRNA.

The target protein may include any type of protein, for example, medically useful proteins including enzymes, hormones, antibodies, and cytokines, or proteins which can accumulate a great amount of nutrients to enhance health of animals including humans, without being limited thereto. Examples of the target protein include interleukin, interferon, platelet-derived growth factors, hemoglobin, elastin, collagen, insulin, fibroblast growth factors, human growth factors, human serum albumins, erythropoietin, or the like, without being limited thereto.

Further, the present disclosure provides a method of producing target protein in an aboveground organ of a plant by transforming the plant using the recombinant plant expression vector. Also, the present disclosure provides target protein produced by the production method. The produced target protein is illustrated as above.

Plant transformation denotes any method of transferring DNA to a plant. The transformation method does not necessarily have a period for regeneration and/or tissue culture. Transformation of plant species is general not only for dicotyledonous plants but also for monocotyledonous plants. In principle, any transformation method may be used to introduce a hybrid DNA sequence of the present disclosure into an appropriate progenitor cell. The method may be properly selected from a calcium/polyethylene glycol method for protoplasts (Krens, F. A. et al., 1982, Nature 296, 72-74; Negrutiu I. et al., June 1987, Plant Mol. Biol. 8, 363-373), an electroporation method for protoplasts (Shillito R. D. et al., 1985 Bio/Technol. 3, 1099-1102), a microscopic injection method for plant components (Crossway A. et al., 1986, Mol. Gen. Genet. 202, 179-185), a gene gun method for various plant components (DNA or RNA-coated) (Klein T. M. et al., 1987, Nature 327, 70), a (non-complete) viral infection method in Agrobacterium tumefaciens mediated gene transfer or transformation of fully ripened pollen or microspores (EP 0 301 316), or the like. A preferable method in accordance with the present disclosure includes Agrobacterium mediated DNA transfer. More preferably, a binary vector technique disclosed in EP A120516 and U.S. Pat. No. 4,940,838 is used.

The “Plant cell” used for plant transformation may be any plant. The plant cell is may include cultured cells, tissue culture, cultured plant organs, or the whole plant, preferably cultured cells, tissue culture, cultured plant organs and more preferably any type of cell culture.

“Plant tissue” may include differentiated or undifferentiated plant tissues, for example, roots, stems, leaves, pollen, seeds, callus tissues, and various types of cells capable of being cultured, including single cells, protoplasts, sprouts, and callus tissue, without being limited thereto. The plant tissue may be a whole plant, an organ culture, a tissue culture, or a cell culture.

Another aspect of the present disclosure provides a method of producing a transformed plant, the method including transforming a plant cell using the recombinant plant expression vector according to the present disclosure, and re-differentiating the transformed plant cell into a transformed plant.

The method includes transforming a plant cell using the recombinant plant expression vector according to the present disclosure, wherein the transformation may be mediated by Agrobacterium tumefiaciens. Further, the method also includes re-differentiating the transformed plant cell into a transformed plant. A method of re-differentiating the transformed plant cell into a transformed plant may be carried out by any method publicly known in the pertinent art.

In addition, the present disclosure provides a transformed plant produced by the method of producing the transformed plant. The plant may preferably be a monocotyledonous plant, more preferably rice, barley, wheat, maize, millet or sorghum, without being limited thereto.

Also, the present disclosure provides a seed obtained from the transformed plant. The seed may preferably be derived from a monocotyledonous plant, and more preferably rice, barley, wheat, maize, millet, or sorghum, without being limited thereto.

Promoters according to the present disclosure may be used for transformation of plants, especially monocotyledonous plants. Particularly, the promoters may make a significant contribution to production of transformed plants in order to express leaf-specific genes in monocotyledonous plants, such as rice.

### BRIEF DESCRIPTION OF DRAWINGS

FIG. 1 illustrates expression of aboveground-specific genes of different tissues of rice;

FIG. 2 is a view of a rice transforming vector;

FIG. 3 illustrates an example of a structure of a promoter according to the present disclosure;

FIG. 4 illustrates expression of GFP in a transformed rice grain;

FIG. 5 illustrates expression of GFP in a transformed seedling;

FIG. 6 illustrates expression of GFP in a transformed rice flower; and

FIG. 7 illustrates comparison of expressed amount of GFP in a seedling using RT-PCR;

FIG. 8 illustrates chloroplast localization of GFP using a research stereomicroscope.

### DETAILED DESCRIPTION

Next, examples of the present disclosure will be described in detail along with comparative examples. However, it should be understood that the present invention is not limited to the following examples.

### EXAMPLE

Materials and Methods

1. Sequence Estimation and Extraction of Promoter

A portion of a promoter was estimated using the International Rice Genome Sequencing Project (IRGSP) started in 1997 and finished in December, 2004 and the TIGR Annotation Data obtained by annotation of genes based on the IRGSP, and the promoter was used to produce a rice transforming vector Annotated BAC was selected, and a sequence from an ATG position to about 2 kbp upstream in a coding sequence (CDS) was assumed as a promoter site and extracted separately as a template for producing a polymerase chain reaction (PCR) primer to isolate a promoter of 1.8 to 2 kb from the 2 kbp promoter.

2. Analysis of of Aboveground Organ-specific Gene by RT-PCR

For analysis of an aboveground organ specific gene, samples were collected from seeds, and leaf, root and flower tissues of 7-day, 30-day and 60-day seedlings. To prepare the samples, the seeds were sterilized with 70% ethanol and a 20% chlorax solution, grown in the dark for five days, and cultivated in a greenhouse. In extraction of whole RNA, RNeasy Plant Mini Kit (Qiagen, Cat. No. 74904) was used. 400 ng of the extracted whole RNA was synthesized into single stranded cDNA (Invitrogen, Cat. No. 18080-051), and PCR was performed with 1 μl of the cDNA synthesis product as a template. The following ubiquitin primers (Ubi) having the following sequences for comparison of amount of used cDNA (loading control) were used during PCR.

Forward primer RbcS3:
(SEQ. ID. No. 3)
5′-TATACAGAGGAGACTCGATTGA-3′
Reverse primer RbcS3:
(SEQ. ID. No. 4)
5′-TACATACACAGCTGATGTTGAC-3′
Forward primer PRK:
(SEQ. ID. No. 5)
5′-GGCATCCTCGCATTTCTTGTA-3′
Reverse primer PRK:
(SEQ. ID. No. 6)
5′-AGAGGTAGGAGCATCCTCAT-3′
Reverse primer RbcS1:
(SEQ. ID. No. 7)
5′-GGTGGCAACTAAGCCGTCAT-3′
Reverse primer RbcS1:
(SEQ. ID. No. 8)
5′-AAGCAGAGCACGGCCGGTAA-3′
Forward primer OsCc1:
(SEQ. ID. No. 9)
5′-ACTCTACGGCCAACAAGAAC-3′
Reverse primer OsCc1:
(SEQ. ID. No. 10)
5′-CTCCTGTGGCTTCTTCAACC-3′
Forward primer OsUbi1:
(SEQ. ID. No. 11)
5′-ATGGAGCTGCTGCTGTTCTA-3′
Reverse primer OsUbi1:
(SEQ. ID. No. 12)
5′-TTCTTCCATGCTGCTCTACC-3′

PCR was performed using a PTC 200 PCR system (MJ research) with 1 μl of cDNA, 2× Taq premix (Solgent. Co. Cat. No. EP051020-T2B6-1), and 4 pmol of each template-specific primer with respect to 20 μl in total at 95° C. for 30 seconds, 55° C. for 30 seconds, and 72° C. for one minute with a cycle of 32.

3. Amplification and Isolation of Promoter

With the estimated 2 kbp promoter sequence as a template, a PCR primer to isolate a promoter having a total size of about 1.8 to 2 kb was designed using Primer Designer 4 (ver. 4.20, Scientific & Educational software). The PCR primer was designed under PCR conditions having GC% of a primer of 40 to 60%, Tm of 55 to 65° C., a salt concentration of 0 mM, and a Mg concentration of 0.15 mM. The PCR primer included a template-specific portion having a length of 20 by and a 5′ adaptor sequence having a length of 12 bp. The adaptor sequence was introduced for position-specific recombination, not for a cloning method using an existing restriction enzyme and DNA ligase. To obtain DNA used as a template during PCR, japonica type rice cultivar Nipponbare was sown and cultivated for three weeks in a greenhouse, after which leaves were cut to extract genomic DNA. The genome DNA was prepared by rapidly freezing the cut leaves in liquid nitrogen, grinding the leaves in a mortar, and isolating using a DNAzo1 solution (molecular research center, Cat. No. DN128). PCR was performed in two stages. A first reaction was to isolate a specific promoter from the rice genome, using a template-specific sequence primer having a total length of 32 by linked to the 12 by adaptor sequence. The primers have the following sequences.

Forward template-specific primer: 5′-AAAAAGCAGGCT-template-specific sequence-3′

Reverse template-specific primer: 5′-AGAAAGCTGGGT- template-specific sequence-3′

In detail, gene-specific primers have the following sequences.

a. RbcS3 promoter primer

Forward primer:
(SEQ. ID. No. 13)
5′-AAAAAGCAGGCTGCGAGGTGCTTAGGCTATTG-3′
Reverse primer:
(SEQ. ID. No. 14)
5′-AGAAAGCTGGGTGCATACAGCTGATCCTTCCAC-3′

b. PRK promoter primer

Forward primer:
(SEQ. ID. No. 15)
5′-AAAAAGCAGGCTGTCTGTTGGCCTACGACAAG-3′
Reverse primer:
(SEQ. ID. No. 16)
5′-AGAAAGCTGGGTCTGAGCATGAAACCTGAAAG-3′

The first PCR was carried out with 50 ng of the genome DNA, 2× Taq premix (Solgent. Co. Cat. No. EP051020-T2B6-1), and 10 pmol of each template-specific primer, with respect to 50 μl in total at 95° C. for one minute, 55° C. for one minute, and 68° C. for two minutes for 30 cycles.

A second PCR was carried out to introduce and amplify a specific adapter sequence (att site) which is needed to introduce a promoter into a transforming vector. A sequence additionally introduced into the promoter had a length of about 29 bp, wherein part (12 bp) of the sequence was attached to overhang the template-specific sequence to perform the first PCR, and then 1/50 (1 μl ) of the PCR product was used as a primer having a whole recombinant sequence (adaptor sequence primer) to perform the second PCR in order to enhance PCR efficiency. Thus, the product obtained the to whole att site for recombination with the rice promoter. The adaptor sequence primers have the following sequences.

attB1 adaptor primer:
(SEQ. ID. No. 17)
5′-GGGGACAAGTTTGTACAAAAAAGCAGGCT-3′
attB2 adaptor primer:
(SEQ. ID. No. 18)
5′-GGGGACCACTTTGTACAAGAAAGCTGGGT-3′

The second PCR was carried out with 1 μl of the first PCR product, 2× Taq premix (Solgent. Co. Cat. No. EP051020-T2B6-1), and 2 pmol of each adaptor primer with respect to 50 μlin total at 95° C. for 30 seconds, 45° C. for 30 seconds, and 68° C. for two minutes with a cycle of 5, and then repeated at 95° C. for 30 seconds, 55° C. for 30 seconds, and 68° C. for two minutes for 20 cycles.

The above PCRs were performed by a method proposed by Invitrogen using a Gateway system (Invitrogen, Cat. No. 12535-029).

4. Cloning of Amplified Promoter

The promoter was introduced into a rice transforming vector using the Gateway system (Invitrogen, Cat. No. 12535-029). First, the amplified promoter was subjected to electrophoresis in a 1% agarose gel, followed by separation of bands on the gel and purification using a Mega-spine agarose gel extraction kit (Intron, Cat. No. 17183). Then, a BP reaction was performed with 5 ul of the purified promoter, 4 ul of a BP clonase mixture, 4 ul of a 5× BP reaction buffer, 300 ng/2 μl of a pDONR vector, and a TE buffer (10 mM Tris/pH 8.0, 1 mM EDTA) with respect to 20 μl in total at 25° C. for 16 hours. Subsequently, the product was mixed with 6 ul of an LR clonase mixture, 1 μl of 0.75 M NaCl, and 450 ng/3 μl of a transforming vector with respect to 30 μlin total, and was subjected to an LR reaction at 25° C. for eight hours. Next, 3 μl of proteinase K was added thereto and reacted at 37° C. for one hour, after which 2 μl of the product was used to transform a DH5 a competent cell. The transformed DH5 a cell was cultured on an LB agar medium including 50 μg/ml spectinomycin in a 37° C. incubator for 12 hours. Then, DNA was extracted from selected cells to identify that the promoters were introduced through PCR, followed by sequencing and BLASTN to identify that the separated promoters were completely introduced.

The rice transforming vector pMJ401 had the following structure: a cassette, to be replaced by a promoter between a right-border sequence and a left-border sequence after recombination, was linked to GFP which is a visible marker gene in a 3′ direction is by a PINII (protease inhibitor II) terminator. The cassette had the att site to perform the BP and LR reactions.

The selective marker genes were produced so that the herbicide resistance genes and bar genes (phosphinothricin acetyltransferase genes), were adjusted by a constitutive promoter, OsCc1 developed by the inventors of the present disclosure (See U.S. Pat. No. 6,958,434), and the genes were linked to a nopalin synthase (NOS) terminator. Further, the genes were combined with an MAR sequence at a right-border sequence end to minimize changes in expression due to an introduced site into a chromosome, thereby measuring activity of the promoter.

5. Agrobacterium-mediated Rice Transformation

A lemma of rice grains (Oryza sativa L. cv Nakdong) was eliminated and washed, gently shaking with 70% (v/v) ethanol for one minute. The washed grains were sterilized, shaking with 20% chlorax for one hour, and washed with sterile water several times. For transformation, the washed rice grains were cultured by the method described by Jang, et al. (see Jang, I-C. et al., Mol. Breeding, 5:453-461, 1999) in a callus induction medium (2N6) for one month to induce callus gemmules. Subsequently, co-cultivation with Agrobacterium prepared by Agrobacterium triple mating was conducted to introduce the above promote-introduced transforming vector into the rice genome, followed by culture in a callus selection medium (2N6-CP) for one month. Then, selectively cultured cells were collected and cultured in a re-differentiation medium (MS-CP) for one to two months, and re-differentiated plants were purified in a greenhouse. Purified new rice plants were treated with a non-selective herbicide, basta, and plants exhibiting an herbicide resistance were selected for a progeny test.

6. Observation of Expressed GFP of Rice Tissues

Expression of the marker genes GFP used to analyze activity of the promoters was identified in tissues of seeds, 5-day seedlings grown in the dark, and flowers. Observation of the expressed genes was performed from a T2 stage where introduction and isolation of the genes are clearly observed. Expression of the GFP in the seeds was observed in gemmules and endosperms of the lemma-eliminated seeds using the LAS is 3000 system (Fuji Photo Film Co.) and a stereomicroscope SZX9-3122 (Olympus, Tokyo, Japan). In the seedlings cultured in the dark, the seeds with the GFP expression identified were sterilized with ethanol and a 20% chlorax solution, and were germinated in an MS-P medium containing PPT (4 mg/l) in the dark for two days in order to identify activity of the selective marker bar genes. The etiolated seedlings grown in the dark were observed with respect to GFP expression using LAS 3000, and then were grown in an incubator chamber for three days, followed by observation of gene expression in young leaves and roots. The process using LAS 3000 was performed under conditions of precision, standard, and exposure time of one second (excitation filter 460nm, barrier filter 510nm). The flowers were collected before earring up, and GFP expression in each tissue was identified using a stereomicroscope SZX9-3122 (Olympus, Tokyo, Japan), observing the flowers before and after elimination of the lemma of the flowers.

7. Analysis of Activity of Promoter through RT-PCR

The whole RNA was extracted separately from young leaves and roots of the seedlings cultured in the dark. The seeds with the expressed GFP identified were sterilized with ethanol and a 20% chlorax solution, and were grown in an MS-P medium containing PPT (4 mg/l) in an incubator chamber in the dark for five days to identify activity of the selective marker bar genes. RNeasy Plant Mini Kit (Qiagen, Cat. No. 74904) was used to extract the whole RNA from the seedling. 400 ng of the extracted whole RNA was synthesized into one strand cDNA (Invitrogen, Cat. No. 18080-051), and a PCR was performed with 2 μl of the cDNA synthesis product as a template. Two types of primers were used in the PCR. A first primer (primer GFP) was to compare relative amount of expressed GFP introduced between promoters, and a second primer (primer OsUbi1) was to compare amount of the used cDNA (loading control), the primers having the following sequences.

Forward primer GFP:
(SEQ. ID. No. 19)
5′-CAGCACGACTTCTTCAAGTCC-3′
Reverse primer GFP:
(SEQ. ID. No. 20)
5′-CTTCAGCTCGATGCGGTTCAC-3′
Forward primer OsUbi1:
(SEQ. ID. No. 11)
5′-ATGGAGCTGCTGCTGTTCTA-3′
Reverse primer OsUbi1:
(SEQ. ID. No. 12)
5′-TTCTTCCATGCTGCTCTACC-3′

The PCR was performed by PTC 200 PCR machine (MJ research) with 2 ul of cDNA, 2× Taq premix (Solgent. Co. Cat. No. EP051020-T2B6-1) and 4 pmol of each template-specific primer with respect to 20 ul in total at 95° C. for 30 seconds, 55° C. for one minute, and 4° C. for 10 minutes with a cycle of 25.

### Example 1

### Analysis of Expression of Aboveground organ specific Genes in Each Rice Tissue

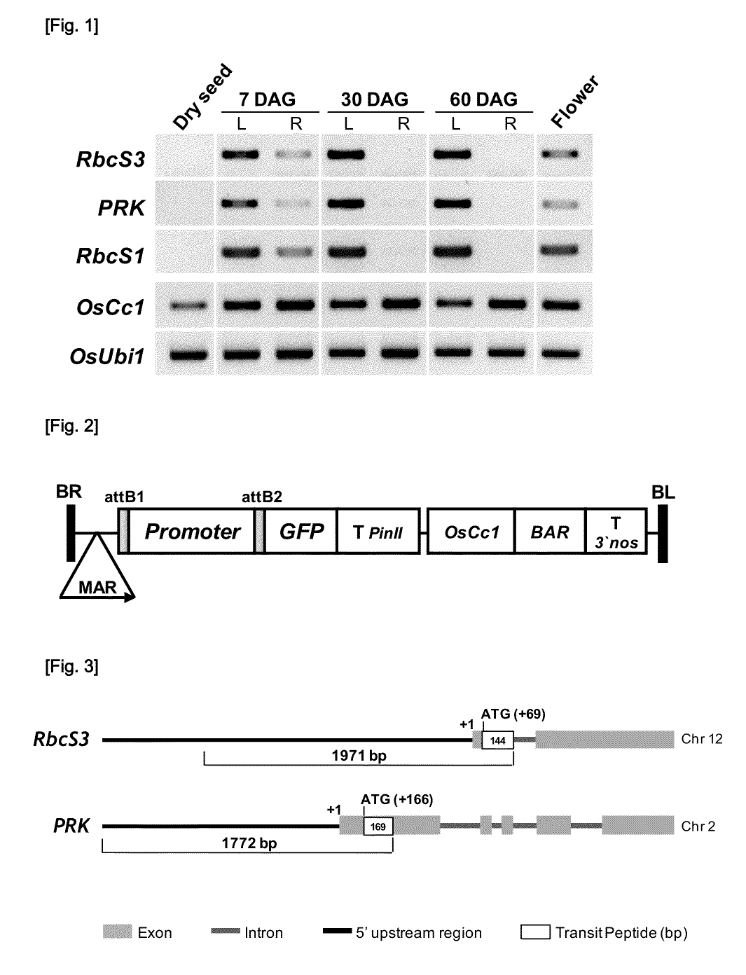

To identify tissue-specific activity of aboveground organ specific promoters of RbcS3 and PRK, samples were collected respectively from seeds, and leaf, root and flower tissues of 7-day, 30-day and 60-day seedlings, and the whole RNA was extracted from each of the samples. The RNA was used as a template to synthesize cDNA, followed by amplification through a PCR and electrophoresis on 2% agarose gel. FIG. 1 illustrates results of expression of two aboveground organ specific genes and known promoters RbcS1 (GenBank accession no. D00643), OsCc1 (GenBank accession no. AF399666), and OsUbi1 (GenBank accession no. AK121590) in each tissue of a rice plant through RT-PCR. FIG. 1 shows that expression of RbcS3 and PRK in each respective tissue of the rice plant is strong in aboveground tissues including leaves and flowers. Further, based on a similar gene expression pattern to the well-known constitutive promoter RbcS1, RbcS3 and PRK are identified as aboveground organ specific genes.

### Example 2

### Production of Rice Transforming Vector and Structure of Promoter

A rice transforming vector for analysis of activity of the promoters was prepared, as shown in FIG. 2. FIG. 2 illustrates a pMJ401 vector, which is a parent vector is to clone the promoters isolated through PCR. AttRl and attR2 sites were recombined (site-specifically recombined) with attL1 and attL2 sequences included in the promoters after BP reaction. After an LR reaction, the promoters were replaced with cassettes, and the attRl and attR2 sequences were replaced by attB1 and attB2 sequences. The respective genes are described as follows:

MAR: Matrix attachment region (1.3 kb), X98408

Cassette B: Transforming cassette B (1.7 kb), invitrogen, Cat. No. 11828-019

GFP: Transformed green fluorescent protein gene (0.74 kb), U84737

TPINII: Protease inhibitor II terminator (1.0 kb), X04118

OsCc1: Cytochrome c promoter (0.92 kb), Af399666

BAR: Phosphinothricin acetyltransferase Genes (0.59 kb), X17220

TNOS: Nopalin synthase terminator (0.28 kb).

FIG. 3 illustrates a structure of the promoters in the rice genome according to the present disclosure.

### Example 3

### Expression of GFP in Transformed Rice Grain

After obtaining progeny until a T3 stage from the transformed rice using each promoter vector, a lemma was eliminated from the rice grains, and GFP expression was observed using a stereomicroscope SZX9-3122 (Olympus, Tokyo, Japan).

FIG. 4 illustrates expression of GFP in the transformed rice grain. Genes of FIG. 4 are described as follows:

NC: Negative control group, Nakdong (Non-transformed seed)

RbcS3: RbcS3 promoter

PRK: PRK promoter

RbcS1: RbcS1promoter

OsCc1: Oryza sativa L. cytochrome C promoter

ZmUbi1: Maize ubiquitin promoter vector.

In the non-transformed control group (negative control group), although a slight background was found in the embryo, GFP was not expressed overall in the grain. In a is positive control group of the OsCc1 promoter and the RbcS1 promoter, although slightly different in extent, GFP was expressed in the embryo with both promoters. Particularly, with the maize ubiquitin promoter, strong expression of GFP was observed not only in the embryo but also in the endosperm.

The two novel promoters (RbcS3 and PRK) according to the present disclosure exhibited weaker GFP expression than the constitutive promoter OsCc1 and the maize ubiquitin promoter, but showed similar GFP expression level to the leaf-specific RbcS1 promoter.

### Example 4

### Expression of GFP in Transformed Seedling

After obtaining progeny until a T3 stage from the transformed rice using each promoter vector, a lemma was eliminated from the rice grains, and GFP expression was observed using a stereomicroscope SZX9-3122 (Olympus, Tokyo, Japan) to ascertain whether the rice grains were homozygotes. After sterilization, the identified grains were grown in an incubator chamber (27° C.) in the dark for five days. Then, GFP expression was observed using the LAS 3000 system (Fuji Photo Film Co.) under conditions of precision, standard, and exposure time of one second (excitation filter 460nm, barrier filter 510 nm). The reason the GFP expression was observed in seedlings grown in the dark is that the GFP expression cannot be properly observed when fluorescence of chlorophyll in the plant causes interference.

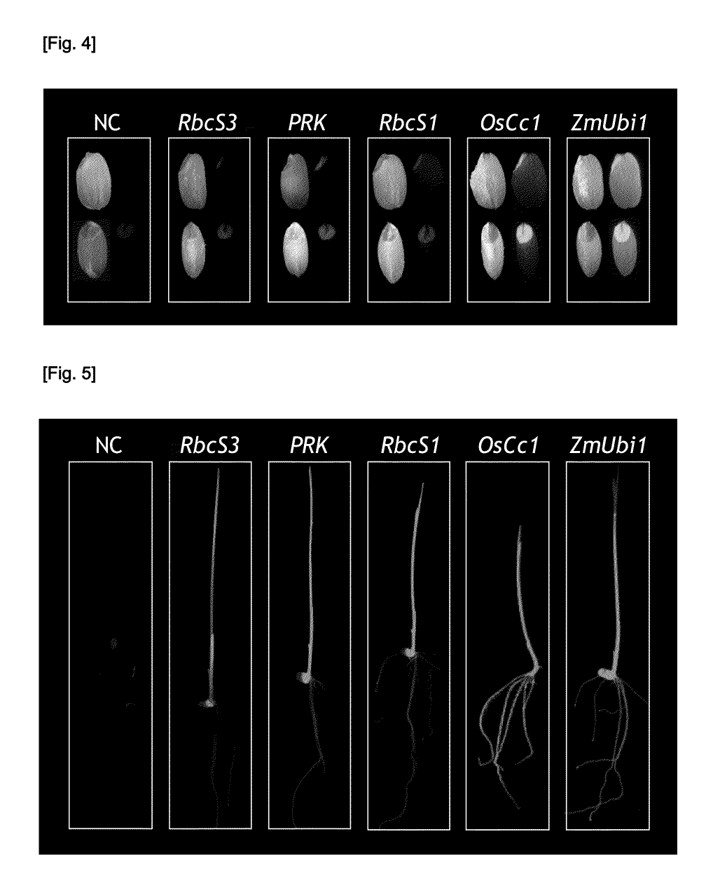

FIG. 5 illustrates expression of GFP in the transformed seedling. Genes of FIG. 5 are described as follows:

NC: Negative control group, Nakdong (Non-transformed seed)

RbcS3: RbcS3 promoter

PRK: PRK promoter

RbcS1: Rbc S1 promoter

OsCc1: Oryza sativa L. cytochrome C promoter

ZmUbi1: Maize ubiquitin promoter vector.

As shown in FIG. 5, in the non-transformed control group (negative control group), although a slight background was found in seeds, GFP was not expressed in the is young leaves and roots. As known conventionally, RbcS1 as a positive control group showed strong GFP expression specifically in leaves, while the OsCc1 promoter and the maize ubiquitin promoter exhibited strong GFP expression in both leaves and roots. The two promoters (RbcS3 and PRK) according to the present disclosure exhibited GFP expression mainly in leaves as the RbcS1 promoter.

### Example 5

### Expression of GFP in Transformed Rice Flower

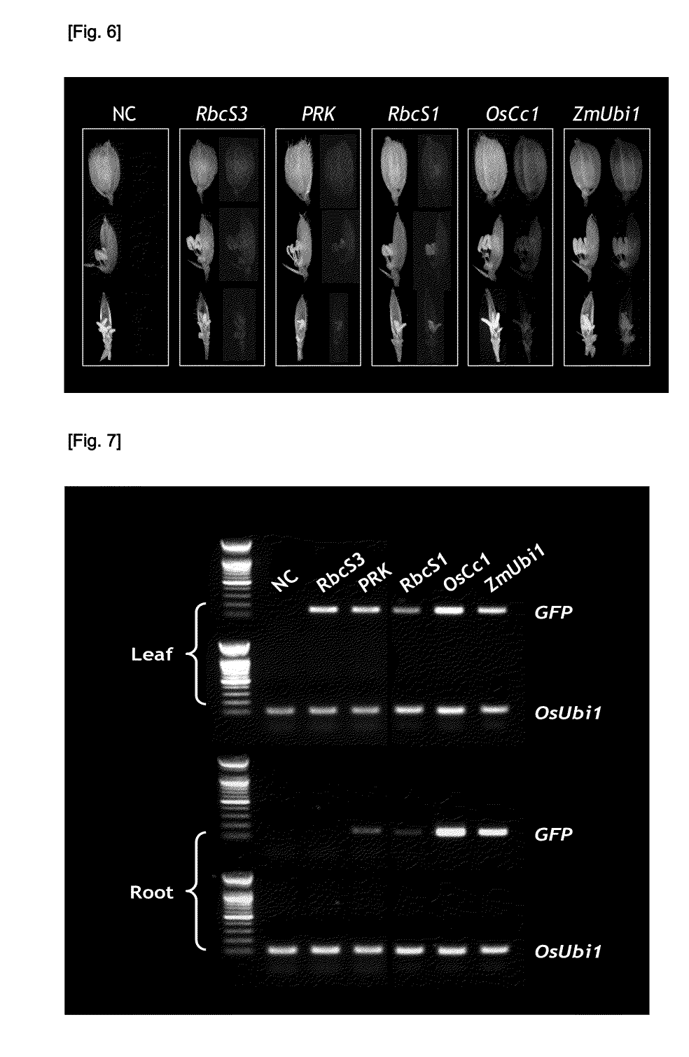

T2 progeny seeds with homozygous GFP expression in seeds and seedlings were cultivated in a greenhouse, and a flower was collected before blooming and subjected to observation of GFP expression in each tissue before and after elimination of a lemma of the flower using a stereomicroscope SZX9-3122 (Olympus, Tokyo, Japan). FIG. 6 illustrates expression of GFP in the transformed rice flower. Genes of FIG. 6 are described as follows:

NC: Negative control group, Nakdong (Non-transformed seed)

RbcS3: RbcS3 promoter

PRK: PRK promoter

RbcS1: Rbc S1 promoter

OsCc1: Oryza sativa L. cytochrome C promoter

ZmUbi1: Maize ubiquitin promoter vector.

With the OsCc1 promoter, GFP was strongly expressed both in rice grains and in leaves and roots of seedlings, but hardly expressed in the flower. With the RbcS 1 promoter, which exhibited leaf-specific expression, GFP was expressed in anthers. With the ZmUbi1 promoter, GFP was strongly expressed in most parts of the flower, such as lema, palea, pistil, anther, or the like, showing that GFP was expressed in the whole plant.

As compared with the control group, although the two promoters (RbcS3 and PRK) according to the present disclosure expressed GFP in all parts of the flower, the expressed amount was not great, and thus the promoters may be considered leaf-specific promoters. Thus, the newly isolated promoters of the present disclosure may be leaf-specific promoters, which have less influence on reproduction than the RbcS promoter known to be a leaf-specific promoter.

### Example 6

### Analysis of Activity (Expression Level of GFP) of Promoter in Transformed Rice Seedling using RT-PCR

Whole RNA was extracted separately from leaves and roots of a seedling cultured in the dark for five days. The RNA was used as a template to synthesize cDNA, followed by amplification through PCR and electrophoresis in a 1.2% agarose gel. 300 ng of a 100 by ladder was used as a marker, and 5 ul of each PCR product was loaded. FIG. 7 illustrates expressed amount of GFP in the seedling, observed using RT-PCR. GFP as a PCR product, obtained by amplification of a GFP primer, was used to compare relative expressed amount of promoter-introduced GFP, and had a length of 142 bp. OsUbi1 as a PCR product, obtained by amplification of a 100 by Ubi primer, was used to compare a cDNA amount (loading control) used as a template.

In FIG. 7, as observed in images of GFP in the rice seedling (See FIG. 5), with the RbcS1 promoter, GFP was very slightly expressed in roots but relatively strongly expressed in leaves, which was considerably slight as compared with the OsCc1 promoter and the ZmUbi1 promoter. With the OsCc1 promoter, GFP was expressed more strongly both in the leaves and in the roots than with the ZmUbi1 promoter.

Thus, the two promoters (RbcS3 and PRK) of the present disclosure were much stronger leaf-specific promoters than RbcS 1. In particular, the RbcS3 promoter induced expression only in the leaves, whereas the RbcS1 promoter and the other promoters induced expression in the roots, although very slightly, as well as in the leaves. The RbcS3 promoter and the PRK promoter of the present disclosure were stronger than the RbcS 1 promoter and similar to the maize ubiquitin promoter. Thus, the two promoters are aboveground organ specific promoters, which realize superior expression amount to the existing RbcS1 promoter. Further, the RbcS3 promoter is leaf tissue-specific, and the PRK promoter is slightly root tissue-specific while inducing stronger expression in leaves, and accordingly the promoters may be selected based on desired expression patterns.

### Example 7

### Chloroplast localization of GFP protein linked to transit is peptide of RbcS3

GFP fluorescence of the rice protoplast which transformed respective constructs was visualized and photographed using a research stereomicroscope (SZX9-3122, Olympus, Tokyo, Japan) equipped with an attachment for fluorescence observations. The constructs consist of three constitutive promoters CaMV 35S (35S), rice GOS2 and PGD1. TP1 and TP3 are transit peptides of RbcS1 and RbcS3, respectively. Images were captured using a C5060-ZOOM digital camera (Olympus). Observations under blue light were carried out using a specific filter set (460-480 nm excitation filters, dichroic mirrors of 485 nm and a 495-540 nm barrier filter).

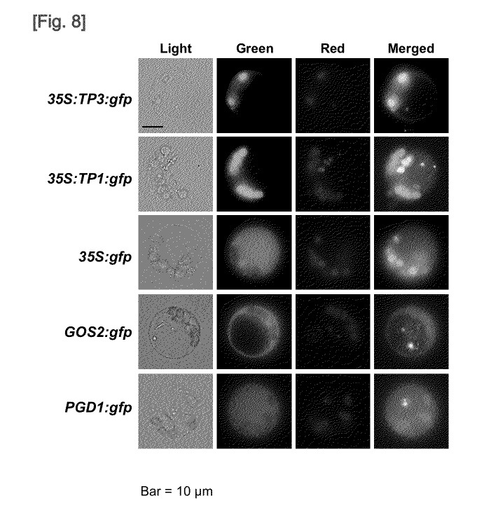

In FIG. 8, the levels of GFP fluorescence were high in the cytoplasm, but relatively low or no in chloroplasts of the 35S:gfp, GOS2:gfp and PGD1:gfp transformed protoplast. GFP fluorescence of 35S:TP3:gfp transformed protoplast which was comparable with that of 35S:TP1:gfp was localized in chloroplasts.

What is claimed is:

1. A promoter comprising at least one sequence having a sequence identification number selected from the group consisting of a sequence identification s number 1 and a sequence identification number 2.

2. The promoter of claim 1, wherein the promoter is an aboveground organ specific promoter for transformation of monocotyledonous plants.

3. The promoter of claim 2, wherein the aboveground organ is a leaf

4. The promoter of claim 1, wherein the promoter comprises a sequence having at least 95% homology with at least one sequence having a sequence identification number selected from the sequence identification number 1 and the is sequence identification number 2.

5. The promoter of claim 1, wherein the promoter comprises a complementary sequence to at least one sequence having a sequence identification number selected from the group consisting of the sequence identification number 1 and the sequence identification number 2.

6. The promoter of claim 2, wherein the monocotyledonous plant comprises rice, barley, wheat, maize, millet, or sorghum.

7. A recombinant plant expression vector comprising the promoter of claim 1.

8. The recombinant plant expression vector of claim 7, wherein the vector is produced by operatively linking a target gene which encodes for a target protein to downstream of the promoter.

9. A method of producing target protein in an aboveground organ by transforming a plant with the recombinant plant expression vector of claim 8.

10. The target protein produced by the method of claim 9.

11. The target protein of claim 10, wherein the target protein comprises at least one selected from the group consisting of interleukin, interferon, platelet-derived growth factors, hemoglobin, elastin, collagen, insulin, fibroblast growth factors, human growth factors, human serum albumins, and erythropoietin.

12. A method of producing a transformed plant comprising:
transforming a plant cell with the vector of claim 7; and
re-differentiating the transformed plant cell into a transformed plant.

13. A transformed plant produced by the method of claim 12.

14. The transformed plant of claim 13, wherein the plant is a monocotyledonous plant.

15. A seed of the plant of claim 14.

16. A seed of the plant of claim 13.


SEQUENCE LISTING (20 sequences)

SEQ ID NO: 1 - DNA, 1971 bp, Oryza sativa
  promoter (1)..(1971): RbcS3 promoter
gcgaggtgcttaggctattgaatatcattatttagagttatatgctgcagatatgtcgaggtgggccgctgatggtgacaactcagtacgagtattcctccgcgcgtgtatataatcctaagttattttcctagggagggtactcctccatatttagccccggttggatggccatgatggattgtcataaggaactcgacaaccagggtggtttctcgaaacaccaggagggcatcgtaagggggtattggctatataattatatagtagataatattgactaaagtgtatgtatgtatattaaaagtatagcaaatgactcttttcttattctttccacttagtctagaattaatatgtatgagaaaagtgaatgcttctgcttagtgtcaccctacccttttggattatattaactcttgtttagactttgattattacaagtaacatataaatattagcttggttctctccataataatctttccaaaggattaaacaaatacgatacccttggaatactctcgggtgaaatgctacaatggtatatccgtgcgcttgcggatgaaatctgtaaccgtaatatactaagagtgtttctgcgtcattgctggaaattatatttttgataatgtcgttaagaataccaacacgcggtatcctacaattaaaaccgcgaagtcatgatgtcacgtaacaaaatttaccttttttagctacggagtacgtgtgtaacgacttgtctttaagagctgctatagataaaatttgccttttttagctacggagtacgtgtgtaacgacttgctttaagagctgctatagatacataattaactgacaggtgttggctgccggaattgttataaaaatctgtcgatagatttttaacaaaataatctgtcaatttttagactacggtttaagttaagattagtgctagggtgctagaaaaatgcaaccgcgcgagagagctggagcctggagggaggaatcgagcgcgtggacatcatgcgctgctgttccaagctacttaccaactgagctagcaggtattgatggttttgttgcaaagatatatttatattaaaattaccgccgttacactatatttacaatgtaaatacactataattaaaaataaaacatttatttattatgatgtaatttttttatctggattgtaacttttctactacttggtgtaatttctgttgttgttaagcccagtagttaactagtgtgtttttttcttcctttttgctagcgtcagataaagttccatggtattagagcaggtacaatagtaggctattagccagctataaacatattttaataagataaaaaataagagataagagaagcggactacagatttatagtcagctgcagcacggactctaagatgcagtgtgtgtatgataggtgtgaccatatattaatagtatagtaagcagctgttatattgtgtgaattgactattagatttagctatatataaattaaagctagtagtgagctatcctattaaacttactcttaacacccgtacttgaatttgatagttttaatctgaggcgtacgtagctatttggtggcttgaaaatctgagtgatttagcagccagccagtcacccgtgctttggatcagcgaacggcagccactctttccacatccccggatcaaccaagcgctgccttccaatgccacctgcgatttggttatttatagacgtaatggattaccacacaggtcctcaagccactgctgcagcgtgactctgctctagcacctcaccaagcagaaagctagagagctagcaatggcgcccaccgtgatggcctcgtcggccacctccgttgctcccttccaggggctcaaatccaccgccgggctccccgtcagccgccgctccaacagcgccggcctcggcagcgtcagcaacggtggaaggatcagctgtatgc

SEQ ID NO: 2 - DNA, 1772 bp, Oryza sativa
  promoter (1)..(1772): PRK promoter
gtctgttggcctacgacaagagccatgctcgttggaaccagaagacagcaacccatctgtctggggatctcatttgtgctttgagacttggaaggaatcttgcactccttggagattcagcttccgccgaacttgggatcaggtgcagtcccaactacaactgctgaacttgggatcaggtgcagtcccaactacaactgcacgttagggactgcacaagatcttgtccattcaatttttcagtggaagatctagattaaatgctacaataaccttgttataataattttgtgctagattgtttgaatgaacagtgtccattaaatttgggctttggatttgcatcccacagccgtagcatgactgtctaagttgcagtcatactggactgcacaggaaccaaattcgtactcttgtgctcaccaatgtttatgtatttttttaacagcaaatacagaaccaaatgaggctgtttatcatatttttcccaggtaataatgttctggtttggcacggtttgtattataagcattgatacaacttgagttgagaatacatgccagaaaatcggcaaacaaataatatacggcacaaacctgacattatatatcatcaatgcataattgattatatcatacggctgaccattaacttgcaagataaaattgcactggaaagtggaaacaaatcgaatttcttcagtttcgtggccgtgggatgttgggcgttggcagccccccagcatatgctttggagatgtcctgcgagtctcggccatctagacatctagtgacatggatgctgacttgtcttggccacacagtgacgcggctgctgatgaagtgatgaggtgctcacctgctgccacgagattacgcgtggactgcgttggtgggctagatgtacatatatggtgcacagcaaacagcaaggttgaagggagcagcatcagcgatatccagcctccagtttccagcctccattccctcatcctcagccagccatccctgcgctgccacgtggcataaccatatctaaattcatgaaaccaccaccaagctttctcccgagcatccctggccatcacaaattcaagcatcgattaatcggcagcacaggaacaagtgcatgtattctccgggacataaaaatgcaggacgtgtaaaataaatctcaaagtacgtccttctcttcgtatcttggcagctagcagacaagcaaaaaagcactgcaactttcagcacataatacttatataaattcagaaattaagtgagaaatgttagaaacatcaccagaaaggatgattgtaagagagccaaaaagcttttcttaacaataaaattcagtgtccatagggagaatgctgacaaaatatgattatttgaaattgaagaactcattttcagttcactataagctgcccttgatatcatcagaaaagaaaggaaaataaggggtaccacatcatcatcatcctctctcctccttatgttctcctagctctatcccctcaattctttcaacctcctctctcttctcatcaagcctcaatcaaaaggcttgaagggaagaagagcaactccagcaggtagaaaggccagaagcaaagatggcgatctccagcctacatgccaccacctccctgcactccccatgcaccaccaacacaagcttcaggcagaatcaggtcatcttcttcaccaccagaagcaacaggaggggtagcacaaggtacggaggagcaagaacctttcaggtttcatgctcag

SEQ ID NO: 3 - DNA, 22 bp, Artificial Sequence
  RbcS3 forward primer
tatacagaggagactcgattga

SEQ ID NO: 4 - DNA, 22 bp, Artificial Sequence
  RbcS3 reverse primer
tacatacacagctgatgttgac

SEQ ID NO: 5 - DNA, 21 bp, Artificial Sequence
  PRK forward primer
ggcatcctcgcatttcttgta

SEQ ID NO: 6 - DNA, 20 bp, Artificial Sequence
  PRK reverse primer
agaggtaggagcatcctcat

SEQ ID NO: 7 - DNA, 20 bp, Artificial Sequence
  RbcS1 forward primer
ggtggcaactaagccgtcat

SEQ ID NO: 8 - DNA, 20 bp, Artificial Sequence
  RbcS1 reverse primer
aagcagagcacggccggtaa

SEQ ID NO: 9 - DNA, 20 bp, Artificial Sequence
  OsCc1 forward primer
actctacggccaacaagaac

SEQ ID NO: 10 - DNA, 20 bp, Artificial Sequence
  OsCc1 reverse primer
ctcctgtggcttcttcaacc

SEQ ID NO: 11 - DNA, 20 bp, Artificial Sequence
  OsUbi1 forward primer
atggagctgctgctgttcta

SEQ ID NO: 12 - DNA, 20 bp, Artificial Sequence
  OsUbi1 reverse primer
ttcttccatgctgctctacc

SEQ ID NO: 13 - DNA, 32 bp, Artificial Sequence
  RbcS3 promoter forward primer
aaaaagcaggctgcgaggtgcttaggctattg

SEQ ID NO: 14 - DNA, 33 bp, Artificial Sequence
  RbcS3 promoter reverse primer
agaaagctgggtgcatacagctgatccttccac

SEQ ID NO: 15 - DNA, 32 bp, Artificial Sequence
  PRK promoter forward primer
aaaaagcaggctgtctgttggcctacgacaag

SEQ ID NO: 16 - DNA, 32 bp, Artificial Sequence
  PRK promoter reverse primer
agaaagctgggtctgagcatgaaacctgaaag

SEQ ID NO: 17 - DNA, 29 bp, Artificial Sequence
  attB1 adaptor primer
ggggacaagtttgtacaaaaaagcaggct

SEQ ID NO: 18 - DNA, 29 bp, Artificial Sequence
  attB2 adaptor primer
ggggaccactttgtacaagaaagctgggt

SEQ ID NO: 19 - DNA, 21 bp, Artificial Sequence
  GFP forward primer
cagcacgacttcttcaagtcc

SEQ ID NO: 20 - DNA, 21 bp, Artificial Sequence
  GFP reverse primer
cttcagctcgatgcggttcac